(19) United States
(12) Patent Application Publication
(10) Pub. No.: US 2026/0159805 A1
(43) Pub. Date: Jun. 11, 2026

(54) LIMOSILACTOBACILLUS MUCOSAE AND DISORDERS REQUIRING A GLP-1 INCREASE

(71) Applicants: INSTITUT NATIONAL DE RECHERCHE POUR L'AGRICULTURE, L'ALIMENTATION ET L'ENVIRONNEMENT, Paris (FR); INSTITUT NATIONAL DES SCIENCES ET INDUSTRIES DU VIVANT ET DE L'ENVIRONNEMENT, Palaiseau (FR); UNIVERSITÉ CLERMONT-AUVERGNE, Clermont-Ferrand (FR); INST ENS SUP RECH ALIMENTATION SANTE ANIMALE SCIENCES AGRONOMIQUES ET ENVIRONNEMENT (VETAGRO SUP), Marcy l'Etoile (FR); ASSISTANCE PUBLIQUE - HÔPITAUX DE PARIS, Paris (FR); UNIVERSITÉ PARIS CITÉ, Paris (FR); UNIVERSITÉ PARIS-SACLAY, Gif-sur-Yvette (FR)

(72) Inventors: Isabelle SAVARY-AUZELOUX, Clermont-Ferrand (FR); Muriel THOMAS, Igny (FR); Christophe CHASSARD, Aurillac (FR); Muriel GIRON, Le Crest (FR); Dominique DARDEVET, Perignat Les Sarlieve (FR); Marianne JARZAGUET, Loubeyrat (FR); Camille MAYEUR, Beaune (FR); Françoise JOLY, Paris (FR); Stéphanie BORNES, Teissiere-Les-Boulies (FR)

(21) Appl. No.: 19/129,197

(22) PCT Filed: Nov. 17, 2023
(86) PCT No.: PCT/EP2023/082290

§ 371 Date: May 12, 2025

(30) Foreign Application Priority Data
Nov. 17, 2022 (FR) FR2211964

(51) Int. Cl. C12N 1/205; A61K 35/00; A61K 36/064; A61P 3/10; A61P 5/00; A61P 21/00; C12R 1/01; C12R 1/23; C12R 1/245; C12R 1/46

(52) U.S. Cl. CPC C12N 1/205; A61K 36/064; A61P 3/10; A61P 5/00; A61P 21/00; A61K 2035/115; C12R 2001/01; C12R 2001/23; C12R 2001/245; C12R 2001/46

(57) ABSTRACT

The present invention relates to a bacterial strain of the species Limosilactobacillus mucosae, or a lysate or culture supernatant thereof, for use in the prevention and/or treatment of disorders requiring an increase in GLP-1 production in a subject in need of GLP-1, the disorders being selected from (i) a disorder related to dysregulation of insulin sensitivity and/or blood glucose concentration and (ii) loss of muscle mass and/or muscle function.

### TECHNICAL FIELD

The present invention relates to new probiotic and prebiotic agents allowing stimulation of the synthesis of GLP-1 by intestinal cells.

In particular, the invention relates to a bacterial strain of the species Limosilactobacillus mucosae, or a lysate or a culture supernatant thereof for use in the prevention and/or treatment of disorders requiring an increase in the level of GLP-1 in a subject in need thereof, said disorders being selected from (i) a disorder linked to dysregulation of insulin sensitivity and/or blood sugar and/or (ii) loss of muscle mass and/or muscle function. It also relates to the non-therapeutic use of a bacterial strain of the species Limosilactobacillus mucosae for maintaining or increasing muscle mass and/or function in subjects in need thereof, in particular in subjects selected from malnourished subjects, elderly subjects, in particular malnourished elderly subjects, and subjects engaged in intense physical exercise. Finally, the invention concerns the bacterial strain Limosilactobacillus mucosae deposited with the CNCM under accession number CNCM I-5661.

### PRIOR ART

The proportion of elderly people (65 years and over) is expected to represent a third of the French population by 2050. This population is very heterogeneous and is characterized, at the extremes, by individuals in good health, active, of good nutritional status, low sarcopenia (age-related loss of muscle mass and function) and generally living at home. Conversely, there exists (at the same age) an undernourished and highly sarcopenic elderly population, generally dependent and living in institutions and in a state of chronic undernutrition (Buckinx et al., Burden of frailty in the elderly population: perspectives for a public health challenge. Archives of public health=Archives belges de santé publique 2015; 73(1): 19). At the same time, in certain Western countries, overweight or obese individuals account for more than half of the population. A part of this population, in addition to an increased susceptibility to developing diabetes or cardiovascular disease, can also present increased muscle wasting, which can reduce the autonomy of these people and above all limit the room for maneuver when treating obesity by means of hypocaloric diets (Barazzoni et al., Sarcopenic Obesity: Time to Meet the Challenge. Obesity Facts. 2018:11(4):294-305). In addition, loss of muscle mass and function can also affect individuals from time to time over the course of their lives, depending on their background and experiences. This is particularly the case for “severely burned” subjects, immobilized patients, cancer patients or even subjects who have undergone intestinal resection or show intestinal malabsorption.

Recent data suggest a causal link between the activity of the intestinal microbiota and various diseases linked to dysregulation of nutrient metabolism and insulin resistance (Marchesi et al. The gut microbiota and host health: a new clinical frontier [Review]. Gut. 2016 February; 65(2):330-9). These data thus suggest the interest in developing probiotic strategies. In addition, comparative physiology studies focus on physiopathological situations in which individuals show metabolic adaptations favorable to the preservation of insulin sensitivity, muscle mass and/or better efficiency of metabolic use of nutrients.

It has been observed that certain lactobacilli were over-represented in individuals who underwent intestinal resection (Mayeur et al. Extensive Intestinal Resection Triggers Behavioral Adaptation, Intestinal Remodeling and Microbiota Transition in Short Bowel Syndrome [Review]. Microorganisms. 2016 Mar. 8; 4(1)). The digestive tract and the microbiota of these patients have the particularity of having adapted metabolically to maintain their energy-nitrogen anabolism despite reduced intestinal food absorption capacity (Gillard et al. Enhanced Ghrelin Levels and Hypothalamic Orexigenic AgRP and NPY Neuropeptide Expression in Models of Jejuno-Colonic Short Bowel Syndrome. Scientific reports. 2016 Jun. 21; 6:28345).

A study of transferring these bacteria to axenic rats showed an increase in the concentrations of plasma peptides such as leptin, ghrelin and GLP-1, suggesting an increase in insulin sensitivity and an increase in the efficiency of energy recovery in these animals (Gillard L, Mayeur C, Robert V, et al. Microbiota Is Involved in Post-resection Adaptation in Humans with Short Bowel Syndrome. Frontiers in Physiology. 2017; 8:224). In fact, GLP-1, a digestive peptide produced by the enteroendocrine cells of the distal ileum and the colon following ingestion of the meal, has numerous metabolic effects including the regulation of insulin secretion by the pancreas (but also of food intake and transit) (Laurindo et al. GLP-1a: Going beyond Traditional Use. Int J Mol Sci. 2022 Jan. 10; 23(2)). GLP-1 analogues are furthermore currently used to improve glucose tolerance in patients, and a role for GLP-1 in Parkinson's disease and the regulation of mood disorders is even reported. GLP1 receptor agonists are frequently used to treat type II diabetes and obesity (Laurindo et al. GLP-1a: Going beyond Traditional Use. Int J Mol Sci. 2022 Jan. 10; 23(2). Their use has also been proposed in many other disorders such as appetite regulation (Aldawsari et al., “The Efficacy of GLP-1 Analogues on Appetite Parameters, Gastric Emptying, Food Preference and Taste Among Adults with Obesity: Systematic Review of Randomized Controlled Trials.” Diabetes, Metabolic Syndrome and Obesity: Targets and Therapy 16 (2023): 575-595), liver diseases (Newsome et al., “A Placebo-Controlled Trial of Subcutaneous Semaglutide in Nonalcoholic Steatohepatitis.” New England Journal of Medicine 384, No. 12 (Mar. 25, 2021): 1113-1124), or degenerative diseases such as Alzheimer's (Femminella et al., “Evaluating the Effects of the Novel GLP-1 Analogue Liraglutide in Alzheimer's Disease: Study Protocol for a Randomised Controlled Trial (ELAD Study).” Trials 20, No. 1 (Apr. 3, 2019): 191) or Parkinson's disease (Athauda et al., “Exenatide Once Weekly versus Placebo in Parkinson's Disease: A Randomised, Double-Blind, Placebo-Controlled Trial.” Lancet (London, England) 390, No. 10103 (Oct. 7, 2017): 1664-1675).

In particular, it has been shown that certain GLP-1 agonists could have a beneficial impact on body composition, particularly on the preservation of muscle mass, including, but not limited to, obese or diabetic individuals (Osaka et al, “Favorable Appendicular Skeletal Muscle Mass Changes in Older Patients With Type 2 Diabetes Receiving GLP-1 Receptor Agonist and Basal Insulin Co-Therapy,” Clinical Medicine Insights: Endocrinology and Diabetes 16 (Jan. 1, 2023): 11795514231161884 or Hong et al., “Amelioration of Muscle Wasting by Glucagon-like Peptide-1 Receptor Agonist in Muscle Atrophy.” J Cachexia Sarcopenia Muscle 10, No. 4 (August 2019): 903-918) or elderly individuals (Abdulla et al., “Glucagon-like Peptide 1 Infusions Overcome Anabolic Resistance to Feeding in Older Human Muscle,” Aging Cell 19, No. 9 (2020): e13202).

If bacteria resident in the distal part of the intestine (colon, distal ileum) are able to stimulate endogenous GLP-1 synthesis, all of the metabolic effects associated with GLP-1 and described above can be expected.

The beneficial effects on humans of certain bacteria of the Lactobacillus genus are known in the art. For example, it is known that L. reuteri, through different mechanisms and metabolites, is capable of controlling body weight and obesity, or improving insulin sensitivity and glucose homeostasis (Abuqwider et al. Limosilactobacillus reuteri in Health and Disease. Microorganisms 2022, 10, 522). Furthermore, it has been demonstrated, for example, that the bacterial strain L. fermentum MG4295 could improve hyperglycemia in mice fed a high-fat diet and that this bacterial strain exhibited properties favorable to its use as a probiotic (Kim et al., Limosilactobacillus fermentum MG4295 Improves Hyperglycemia in High-Fat Diet-Induced Mice. Foods. 2022, 11, 231).

Furthermore, it is known in the art that certain strains of the species L. mucosae could present probiotic potential, for example on lipid metabolism and in particular on the control of hyperlipemia (CN 111979145 A1), but also in the treatment of memory disorders, learning disorders, mental disorders and inflammatory diseases (EP 3715449 A2) or even in protection against certain cardiovascular diseases (Ryan et al., BMC Microbiology (2019) 19:33).

However, to the knowledge of the inventors, it has never been suggested in the past that a bacterial strain of the species L. mucosae could present probiotic properties allowing the prevention and/or treatment of disorders requiring an increase in production of GLP-1 in a subject in need thereof.

Thus, there remains a need to propose new probiotic compositions making it possible to stimulate the production of GLP-1, depending on the subjects concerned, for (i) preventing or treating a disorder linked to dysregulation of insulin sensitivity and/or blood sugar or (ii) preventing or treating loss of muscle mass and/or loss of muscle function.

### Presentation of the Invention

Surprisingly, it is shown in the examples that a bacterial strain of the species Limosilactobacillus mucosae, or a lysate or culture supernatant thereof, induces, or increases, the production of the GLP-1 protein, in particular by the intestinal cells of a human subject.

According to a first object, the invention relates to a bacterial strain of the species Limosilactobacillus mucosae, or a lysate or a culture supernatant thereof, for use in the prevention and/or treatment of disorders requiring an increase in the production of GLP-1 in a subject in need thereof, said disorders being selected from (i) a disorder linked to dysregulation of insulin sensitivity and/or blood sugar and (ii) loss of muscle mass and/or muscle function.

According to a particular embodiment, the subject in need thereof is a subject in whom administration of GLP-1 receptor agonists is indicated. In particular, a subject in whom administration of GLP-1 receptor agonists is indicated is selected from the list consisting of insulin-resistant subjects such as diabetic subjects, overweight subjects, in particular obese subjects, subjects having undergone metabolic surgery, subjects seeking to regulate their appetite, subjects suffering from liver diseases, subjects suffering from cardiovascular diseases, in particular suffering from cardiovascular diseases linked to diabetes, subjects suffering from inflammation, in particular inflammation linked to diabetes and/or metabolic syndrome, subjects with sarcopenia, subjects with cachexia; and subjects suffering from neurodegenerative diseases.

According to one embodiment, the disorder is a disorder linked to dysregulation of insulin sensitivity and/or blood sugar selected from prediabetes, type 1 diabetes and type 2 diabetes.

According to one embodiment, the disorder is a loss of muscle mass and/or function and in that the subject in need thereof is selected from elderly subjects with sarcopenia, overweight and obese subjects with sarcopenic obesity placed on a diet, diabetic or prediabetic subjects and/or subjects with cachexia, in particular linked to cancer, to inflammatory bowel disease or to chronic obstructive pulmonary disease, to immobilization, to “severe burn” status, sepsis or having suffered a viral infection, to convalescence and/or to immobilization, and/or to intestinal resection or intestinal malabsorption.

In particular, the bacterial strain is selected from the strain of the species L. mucosae deposited with the CNCM under accession number CNCM I-5661, the strain of the species L. mucosae deposited with the DSM under accession number DSM 13345, the strain of the species L. mucosae deposited with the DSM under accession number DSM 13346, the strain of the species L. mucosae deposited with the DSM under accession number DSM 102820, or a mixture thereof, and in particular is the strain of the species L. mucosae deposited with the CNCM under accession number CNCM I-5661.

According to one embodiment, the bacteria of the species L. mucosae are in a living or dead form, preferably in a living form.

In particular, the bacterial strain can be included in a composition comprising a physiologically acceptable medium, in particular in an oral composition, and more particularly in an oral composition selected from the group consisting of a food product, a beverage, a pharmaceutical product, a nutraceutical, a food additive, a food supplement, a dairy product and live biotherapeutic products (LBPs).

In particular, the composition further comprises one or more other probiotic bacterial strains, in particular selected from the species Bifidobacterium longum, Bifidobacterium lactis, Bifidobacterium breve, Bifidobacterium infantis, Bifidobacterium adolescentis, Lactobacillus acidophilus, Lactobacillus casei, Lactobacillus salivarius, Lactobacillus johnsonii, Lactobacillus salivarius, Lactococcus lactis, Enterococcus faecium, Enterococcus faecalis, Saccharomyces cerevisiae, Saccharomyces boulardii, Faecalibacterium prausnitzii, Akkermansia muciniphila, Blautia faecis, Faecalibacterium prausnitzii, Streptococcus Thermophilus or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus johnsonii NCC533 (CNCM 1-1225), Enterococcus faecium SF 68 (NCC2768; NCIMB10415), Lactobacillus casei (CNCM I-5662 and CNCM I-5663), Streptococcus thermophilus (CNCM I-5334) and combinations thereof.

According to a particular embodiment, the composition is free of any other bacteria of the Lactobacillus species.

In particular, the composition further comprises one or more prebiotics.

According to a second object, the invention relates to the non-therapeutic use of a bacterial strain of the species Limosilactobacillus mucosae, or a lysate or a culture supernatant thereof, in particular as defined according to the invention, for maintaining or increasing muscle mass and/or function in subjects in need thereof, in particular in subjects selected from malnourished subjects, elderly subjects, in particular malnourished elderly subjects, and subjects engaged in intense physical exercise.

Finally, the invention concerns the bacterial strain Limosilactobacillus mucosae deposited with the CNCM under accession number CNCM I-5661.

### BRIEF DESCRIPTION OF THE DRAWINGS

FIG. 1 shows the concentration of GLP-1 in the supernatant of intestinal neuroendocrine cells from SCT-1 mice after 4 hours of incubation in the presence of the various bacteria and conditions tested. The x-axis shows the different conditions tested, i.e. from left to right: strain 1 (L. casei 1), strain 2 (L. casei 2), strain 3 (L. camelliae), strain 4 (L. salivarius), strain 5 (L. rhamnosus), strain 6 (L. reuleri), strain CNCM I-5661 (L. mucosae) and control (negative—in a sterile cell medium). The y-axis shows the GLP-1 concentration in pg/mL.

FIG. 2 shows the concentration of GLP-1 in the supernatant of intestinal neuroendocrine cells from SCT-1 mice as a function of incubation time in the presence of the strain CNCM I-5661 (L. mucosae). The x-axis shows the incubation time, i.e. from left to right: 3 hours of incubation, 4 hours of incubation and 5 hours. The y-axis shows the GLP-1 concentration in pg/mL.

FIG. 3 shows the concentration of GLP-1 in the supernatant of intestinal neuroendocrine cells from SCT-1 mice after 3 hours of incubation in the presence of different bacteria of the species L. mucosae at different bacterial concentrations. The x-axis shows the different conditions tested, i.e. from left to right: control (negative control, sterile cell medium), strain Lacticaseibacillus casei (strain A-negative control, ˜5.10⁹ CFU/mL), strain CNCM I-5661 (strain B—positive control, ˜5.10⁹ CFU/mL), strain CNCM I-5661 diluted 1/10 (strain B diluted—˜5.10⁸ CFU/mL), strain DSM 13345 (strain C—˜2.10⁹ CFU/mL), strain DSM 13345 diluted 1/10 (strain C diluted—˜2.10⁸ CFU/mL), strain DSM 13346 (strain D—˜4.10⁹ CFU/mL), strain DSM 13346 diluted 1/10 (strain D diluted—˜4.10⁸ CFU/mL), strain DSM 102820 (strain E—˜5.10⁹ CFU/mL), and strain DSM 102820 diluted 1/10 (strain E diluted—˜5.10⁸ CFU/mL). The y-axis shows the GLP-1 concentration in pg/mL. Significant difference (P<0.05). Same comments as above, there is a clear difference between the total absence of secretion detected (for the negative control conditions) and the detectable secretion of GLP-1 under the conditions with L. mucosae bacteria, all strains combined, but no statistics were calculated (insufficient data), and this did not seem necessary in view of the results.

FIG. 4 shows the weight of the muscles of the hind legs of rats (in mg/g of rat weight) for different groups of aged rats (20 months) after one month of study. The groups of rats studied are as follows from left to right, on the x-axis: rats fed ad libitum (AL), rats restricted to 75-80% of ad libitum intake (R), rats restricted and supplemented with L. mucosae of the strain CNCM-15661 (R+L. mucosae I5561), rats restricted and supplemented with L. casei (R+L casei).

### DETAILED DESCRIPTION

The inventors carried out in-depth research to identify the capacity of a species of bacteria, the species Limosilactobacillus mucosae, to treat and/or prevent disorders requiring an increase in the production of GLP-1 in subjects in need thereof.

In fact, the inventors unexpectedly showed that strains of Limosilactobacillus mucosae were capable of stimulating the synthesis of GLP-1 by intestinal cells in vitro, whereas other strains of distinct lactobacilli species were not capable of inducing this synthesis of GLP-1 by intestinal cells. Furthermore, Limosilactobacillus mucosae showed a good capacity for resistance to environmental conditions encountered in the intestine (acidic pH of the stomach and bile salts) as well as a good capacity for adhesion to intestinal cells. Limosilactobacillus mucosae is abundant in patients with short bowel syndrome (Joly et al, Biochemistry 2010; PMID: 20172013) and is capable of colonizing a virgin digestive tract (axenic animal) after fecal transfer (Gillard et al; Front Physiol. 2017); this indicates that this species is a gut commensal. This capacity of Limosilactobacillus mucosae to increase the synthesis of the intestinal peptide GLP-1 and to be present in the digestive tract is important because this peptide has numerous metabolic pleiotropic effects in the host; in particular, it improves insulin secretion by the pancreas, promotes glucose tolerance, and therefore optimizes/improves the energy and nitrogen metabolism of the host. Finally, the inventors showed that administration of a strain of Limosilactobacillus mucosae made it possible to increase muscle mass in a sarcopenic rodent model.

Limosilactobacillus mucosae

The present invention relates to the use of a bacterial strain of the species Limosilactobacillus mucosae, or a lysate or a culture supernatant thereof.

For the purposes of the present invention, the term “lysate” is used interchangeably to designate the entire lysate obtained by lysis of the microorganism concerned or only a fraction thereof.

Dead bacterial cells can be obtained by any method known to the person skilled in the art.

The bacterial cell lysate is made up of all or part of the intracellular biological constituents and the constituents of the cell walls and membranes. It contains in particular the cellular cytoplasmic fraction containing enzymes such as lactic acid dehydrogenase, phosphatases, phosphoketolases and transaldolases. By way of illustration, the constituents of the cell walls are in particular peptidoglycan, murein or mucopeptide and teichoic acid, and the constituents of the cell membranes are composed of glycerophospholipids.

A bacterial cell lysate can be obtained by different technologies, such as for example by exposing bacterial cells to osmotic shock, thermal shock, or ultrasound. More particularly, this lysate can be obtained according to the technology described in U.S. Pat. No. 4,464,362.

“Culture supernatant” refers within the meaning of the present invention to the culture medium in which the bacterial strains were present during their cultivation, which can also be referred to by the term “extracellular medium” in the present description. In particular, the culture supernatant can include the metabolites produced and secreted by the bacteria, also called extracellular metabolites. Examples of metabolites are, for example, peptides, glycopeptides or lipopeptides produced by the bacteria. The culture supernatant can be crude or can have undergone one or more steps to filter it, concentrate it, freeze-dry it, heat it, etc. These techniques for transforming the culture supernatant of a bacterium are known to the person skilled in the art.

The composition of the culture supernatant varies depending on the conditions and cell culture methods of the bacterial strain. It chiefly comprises extracellular metabolites when the biomass is growing or is only active, but also potentially intracellular metabolites for cultures at the end of the stationary phase and when microbial lysis is significantly greater. A portion of the cells die and others lose their membrane integrity during lysis, as a result of which the supernatant will include cell walls from dead cells but also intracellular metabolites.

Furthermore, the culture supernatant can also include bacterial cells of the species Limosilactobacillus mucosae, in living or dead form.

Limosilactobacillus mucosae is a rod-shaped bacterial species of lactic acid bacteria first isolated from pig intestines. It exhibits mucus adhesion activity.

Limosilactobacillus mucosae is a gram-positive bacterium that is an obligate anaerobe, which, however, can multiply to a reduced extent in the presence of oxygen. This bacterial species is described in particular by Roos et al. (2000, International Journal of Systematic and Evolutionary Microbiology, Vol. 50 (1): 251-258). This bacterial species is described in the human microbiota of patients with short bowel syndrome (Drastic changes in fecal and mucosa-associated microbiota in adult patients with short bowel syndrome. Joly F, Mayeur C, Bruneau A, Noordine M L, Meylheuc T, Langella P, Messing B, Duée P H, Cherbuy C, Thomas M. Biochemistry. 2010 July; 92(7):753-61)

Concerning the designation of certain probiotics, and in particular bacteria of the Lactobacillus genus, it is important to note the recent changes in their taxonomic classification, as reported in the article by Zheng et al. (2020, Int. J Syst Evol Microbiol, Vol. 70:2782-2858). Thus, certain probiotics of the genus Lactobacillus documented in the previously published literature with these taxonomic changes are now designated according to the new nomenclature in force.

Thus, the bacterial species known today as Limosilactobacillus mucosae can also be specified in the literature as Lactobacillus mucosae.

A bacterial strain of L. mucosae suitable according to the invention can be derived from the feces of subjects suffering from short bowel syndrome.

According to a particular embodiment, a bacterial strain according to the invention is selected from the strain of the species L. mucosae deposited with the CNCM under accession number CNCM I-5661, the strain of the species L. mucosae deposited with the DSM under accession number DSM 13345, the strain of the species L. mucosae deposited with the DSM under accession number DSM 13346, the strain of the species L. mucosae deposited with the DSM under accession number DSM 102820, or a mixture thereof, and in particular is the strain of the species L. mucosae deposited with the CNCM under accession number CNCM I-5661.

According to a particular embodiment, the bacterial strains of the species L. mucosae are in a living or dead form, preferably in a living form.

Within the meaning of the invention, a “dead bacterial strain” refers to a bacterial cell which is definitely no longer capable of multiplying and consequently of forming a colony in culture. Unlike a lysate, a dead bacterial strain can retain all of its membrane integrity.

A dead bacterial strain can be obtained by any known cell death method. According to a particular embodiment of the invention, the dead bacterial cells according to the invention can in particular be obtained by exposure to high heat (for example by exposure to a UHT protocol).

Thus, for the purposes of the present invention, and in particular in the examples of the present text, the term “living,” unless otherwise indicated, designates living bacterial cells, including stabilized bacterial cells, i.e. viable and revivable bacterial cells (by all known stabilization methods, for example by freezing, freeze-drying or spray drying).

### Compositions

The present description also relates to a composition comprising a bacterial strain of the species Limosilactobacillus mucosae, or a lysate or a culture supernatant thereof.

Thus, according to a particular embodiment, a bacterial strain according to the invention is included in a composition comprising a physiologically acceptable medium.

The term “physiologically acceptable medium” is intended to designate a medium which is compatible with the organism of the individual to whom said composition must be administered. This can be, for example, a non-toxic solvent such as water. The medium can also be a food, in particular when the bacterial strain according to the invention is included in a nutritional composition, as defined below. The medium can also be a mucus, such as snail mucus. A mucus suitable as a medium according to the present invention can in particular be obtained as described in Gillard et al. (Enhanced Ghrelin Levels and Hypothalamic Orexigenic AgRP and NPY Neuropeptide Expression in Models of Jejuno-Colonic Short Bowel Syndrome, Sci Rep. 2016 Jun. 21; 6:28345).

In particular, said medium is compatible with oral administration.

A composition as described can be a nutritional composition.

For example, an athletic subject in whom it is necessary to maintain or increase muscle mass does not present any pathology and only requires nutritional supplementation. According to another example, the composition according to the present description can be a nutritional composition intended for undernourished elderly people or overweight people, undernourished for example because they are on a diet.

The present description also relates to a pharmaceutical composition comprising a bacterial strain of the species L. mucosae, or a lysate or a culture supernatant thereof, which is intended for subjects presenting, or likely to present, a pathology on which said composition has a prophylactic or therapeutic effect. By way of example, a composition according to the present description consists of a pharmaceutical composition intended to prevent or treat dysregulation of insulin sensitivity and/or blood sugar, as in the case of diabetes or prediabetes.

However, unless specifically indicated, a composition comprising a bacterial strain of the species L. mucosae does not differ in its general characteristics set forth in the description depending on whether it is a nutritional composition or a therapeutic composition. Thus, essentially, a pharmaceutical composition is distinguished from a nutritional composition by the fact that the pharmaceutical composition has, in the subject to whom it is administered, a preventive effect and/or a therapeutic effect for a disease. Each of these compositions also meets its own regulations and is distinguished by its mode of action. In fact, pharmaceutical compositions exert a “pharmacological, immunological or metabolic action”; while nutritional compositions have a “nutritional or physiological” effect.

In certain embodiments of a composition according to the description, the bacterial strain of the species L. mucosae is used as the only bacterial strain present in the composition.

In certain other embodiments of a composition according to the description, the bacterial strain of the species L. mucosae is combined with bacteria of one or more other probiotic bacterial strains, which includes commensal probiotic bacterial strains.

Non-limiting examples of probiotics include strains of bacteria belonging to the following genera: Bifidobacterium, Lactobacillus, Lactococcus, Enterococcus, Streptococcus, Kluyveromyces, Saccharomyces, Candida, Blautia, Faecalibacterium, Akkermansia, and combinations thereof.

Probiotics can be selected from the group composed of the following bacterial species: Bifidobacterium longum, Bifidobacterium lactis, Bifidobacterium breve, Bifidobacterium infantis, Bifidobacterium adolescentis, Lactobacillus acidophilus, Lactobacillus casei (now Lacticaseibacillus casei), Lactobacillus paracasei (now Lacticaseibacillus paracasei), Lactobacillus salivarius (now Ligilactobacillus salivarius), Lactobacillus lactis (now Lactobacillus delbrueckii subsp. Lactis), Lactobacillus rhamnosus (now Lacticaseibacillus rhamnosus), Lactobacillus johnsonii, Lactobacillus plantarum (now Lactiplantibacillus plantarum subsp. Plantarum), Lactococcus lactis, Enterococcus faecium, Enterococcus faecalis, Saccharomyces cerevisiae, Saccharomyces boulardii, Akkermansia muciniphila, Blautia faecis, Faecalibacterium prausnitzii, Streptococcus thermophilus or mixtures thereof.

According to a particular embodiment, a composition as described further comprises one or more other probiotic bacterial strains, in particular selected from the species Bifidobacterium longum, Bifidobacterium lactis, Bifidobacterium breve, Bifidobacterium infantis, Bifidobacterium adolescentis, Lactobacillus acidophilus, Lactobacillus casei, Lactobacillus salivarius, Lactobacillus johnsonii, Lactobacillus salivarius, Lactococcus lactis, Enterococcus faecium, Enterococcus faecalis, Saccharomyces cerevisiae, Saccharomyces boulardii, Faecalibacterium prausnitzii, Akkermansia muciniphila, Blautia faecis, Streptococcus Thermophilus or mixtures thereof, preferably selected from the group consisting of Bifidobacterium longum NCC3001 (ATCC BAA-999), Bifidobacterium longum NCC2705 (CNCM I-2618-cited in CA 2761573 A1), Bifidobacterium longum NCC490 (CNCM I-2170-cited in WO 2006037922 A1), Bifidobacterium lactis NCC2818 (CNCM I-3446-cited in WO 2008116916 A1), Bifidobacterium breve strain A, Lactobacillus johnsonii NCC533 (CNCM 1-1225-cited in WO 2017060468), Enterococcus faecium SF 68 (NCC2768; NCIMB10415), Lactobacillus casei (CNCM I-5662 and CNCM I-5663-cited in Giron et al., Front Nutr. 2022; 9:928798, published online 2022 Aug. 10), Streptococcus thermophilus (CNCM I-5334-cited in EP 3826655 B1) and combinations thereof.

In a particular embodiment, the composition is free of any other bacteria of the Lactobacillus species.

In some embodiments, the composition also comprises one or more prebiotics. The term “prebiotic” is used in its conventional sense in the prior art. Prebiotics consist of food substances that promote the growth of probiotic bacteria, including bacteria included in the microbiota.

Non-limiting examples of prebiotics include: oligosaccharides optionally containing fructose, galactose, mannose; dietary fibers, in particular fermentable fibers, soy fibers; inulin; human milk oligosaccharides (HMO); polyphenols; chicory, mucus, and combinations thereof. The preferred prebiotics are fructo-oligosaccharides (FOS), galacto-oligosaccharides (GOS), isomalto-oligosaccharides (IMO), xylo-oligosaccharides (XOS), arabino-xylo-oligosaccharides (AXOS), mannan-oligosaccharides (MOS), soy oligosaccharides, glycosyl sucrose (GS), lactosucrose (LS), lactulose (LA), palatinose-oligosaccharides (PAO), malto-oligosaccharides, resistant starches, gums and/or hydrolysates thereof, pectins and/or hydrolysates thereof, or combinations thereof.

The prebiotics can also include peptides, proteins and complex secretions of peptides, sugars, or sulfur such as mucus (intestinal mucus for example). In pigs, a positive effect of mannan oligosaccharides and L. mucosae was tested (PMID: 34879142; J Anim Sci. 2021 Dec. 1; 99(12)). It has also been shown that a high-fiber diet can induce the culture of L. mucosae (Lactobacillus Mucosae Strain Promoted by a High-Fiber Diet. Microorganisms. 2020 Aug. 12; 8(8):1225. PMID: 32806628)

In certain embodiments, the composition comprises the combination of a probiotic and a prebiotic, which is then referred to as “symbiotic.” The term “symbiotic” is used in its conventional sense in the prior art. The aim of symbiotics is to improve the survival of the probiotic and increase its biological properties.

Non-limiting examples of symbiotics include the combinations bifidobacteria/fructo-oligosaccharides, lactobacilli/lactilol or even bifidobacteria/galacto-oligosaccharides.

In some embodiments, the composition additionally comprises one or more vitamins. The vitamins can be folic acid, vitamin B12 and vitamin B6, especially folic acid and vitamin B12, especially folic acid. In some embodiments, the composition includes one or more vitamins that are fat-soluble, for example one or more vitamins from among vitamin A, vitamin D, vitamin E and vitamin K or water-soluble, such as vitamin C.

In some embodiments, the composition includes one or more polyphenols, such as flavanols, flavanones, flavonols, hydroxycinnamic acids, and anthocyanins.

In some embodiments, the composition further comprises one or more minerals. The minerals can be selected from sodium, potassium, chloride, calcium, phosphate, magnesium, iron, zinc, copper, selenium, manganese, fluorine, iodine, chromium or molybdenum. Minerals are usually added in the form of salt. Minerals can be added alone or in combination.

In some embodiments, a composition according to the present description generally includes supports or vehicles. “Supports” or “vehicles” refer to materials suitable for administration and include any material known in the prior art, such as, for example, any liquid, gel, solvent, liquid diluent, solubilizing agent or other, which is non-toxic and which does not interact with the components of the composition in a harmful manner. Examples of nutritionally acceptable carriers include, for example, water, saline solutions, alcohols, silicones, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, monoglycerides and diglycerides of fatty acids, fatty acid esters of petroleum, hydroxymethylcellulose, polyvinylpyrrolidone, etc.

In certain embodiments, the composition further comprises any other ingredient or excipient known to be used in the type of composition in question. Non-limiting examples of such ingredients include: proteins, amino acids, carbohydrates, oligosaccharides, lipids, nucleotides, nucleosides, other vitamins, minerals, bacterial metabolites, bio-active molecules and other micronutrients.

For example, a bacterial strain according to the invention can be combined with proteins in order to limit muscle wasting of the subject to whom it is administered.

According to another example, a bacterial strain according to the invention can be combined with n-3 polyunsaturated fatty acids in order to increase the insulin sensitivity of the subjects.

In certain embodiments, the composition contains a source of carbohydrates, for example in the form of prebiotics, or prebiotics when they are present in the composition. Any source of carbohydrates typically found in infant formula, such as lactose, sucrose, maltodextrin, starch, and mixtures thereof, can be used, although the preferred source of carbohydrate is lactose.

In some embodiments, a composition according to the present description consists of a nutritional composition.

In some embodiments, the nutritional composition is selected from complete food compositions, food supplements, nutraceutical compositions and others. The composition of the present description can be used as a food ingredient and/or animal feed ingredient. The food ingredient can be in the form of a solution or a solid, depending on the use and/or the method of application and/or the method of administration. As used herein, the term “food” refers to liquid (i.e., beverage), solid, or semi-solid dietary compositions, particularly total food (food replacement) compositions, which do not require additional nutrients or food supplement compositions. Food supplement compositions do not completely replace the provision of nutrients by other means. As used in the present description, the term “food ingredient” includes a formulation that is or can be added to functional foods or foodstuffs as a food supplement. “Nutritional food” or “nutraceutical” or “functional” refers to a foodstuff which contains ingredients having beneficial effects on health or capable of improving physiological functions. “Food supplement” refers to a foodstuff intended to supplement a normal diet. A food supplement is a concentrated source of nutrients or other substances that have a nutritional or physiological effect when taken alone or in combination in small quantities. According to the present description, “functional food” is used to designate foodstuffs and corresponding products to which importance is attributed not only because of their nutritional and taste value but also because of the presence of ingredients having beneficial physiological effects.

In some embodiments, the composition is a fermented dairy product or a milk-based product, which is preferably administered or ingested orally one or more times per day. Fermented dairy products include milk-based products, such as (but not limited to) desserts, yogurts, yogurt drinks, cottage cheese, kefir, fermented milk drinks, buttermilk, cheeses, salad dressings, low-fat spreads, cream cheese, soy drinks, ice cream, etc.

In certain embodiments, the composition is a fermented product based on plant matrices.

As a variant, in some embodiments, the nutritional and/or nutritional supplement compositions can be non-dairy or non-fermented dairy products. Unfermented dairy products can include ice cream, nutritional bars and seasonings, and others. Non-dairy products can include powdered drinks and nutritional bars, etc. The products can be manufactured using known methods, such as adding an effective amount of a bacterial strain of the species L. mucosae, or a combination of bacteria including the bacterial strain of the species L. mucosae, to a food base, such as skimmed milk or milk or a milk-based composition, and carrying out fermentation using any known technique. In some embodiments, the composition is a beverage that can be a functional beverage or a therapeutic beverage, a thirst-quenching beverage, or a conventional beverage. By way of example, the composition according to the present description can be used as an ingredient for carbonated drinks, a fruit juice or a drink comprising whey proteins, teas, cocoa drinks, milk drinks, yogurts, including drinking yoghurts, cheeses, ice creams, ice pops and desserts, confectionery, biscuits, cakes and cake mixes, snacks, healthy foods and drinks, icings, acidified soy drink/juice, aseptic/reduced chocolate drink, bar mixes, powdered drink mixes, calcium-enriched soy milk and chocolate, calcium-enriched coffee drink.

In some embodiments, the composition includes any other ingredient or excipient known to be used in the type of composition in question. Non-limiting examples of such ingredients include: proteins, amino acids, carbohydrates, oligosaccharides, lipids, prebiotics or probiotics, nucleotides, nucleosides, other vitamins, minerals and other micronutrients,

In certain other embodiments, bacterial strains of the species L. mucosae, optionally in combination with probiotic bacteria of one or more other strains, are administered to the subject in the form of a pharmaceutical composition, which could correspond to a product of the Live Biotherapeutic Product (LBP) type, reference: Front Med (Lausanne) Rouanet et al., 2020 Jun. 19; 7:237. doi:10.3389/fmed.2020.00237. For example, the bacteria of interest can be combined with pharmaceutically acceptable excipients, and optionally sustained-release matrices such as biodegradable polymers, to form therapeutic compositions. The terms “pharmaceutically” or “pharmaceutically acceptable” refer to molecular entities and compositions that do not cause an adverse, allergic or other reaction when administered to a mammal, particularly humans, as applicable. A pharmaceutically acceptable carrier or excipient refers to a non-toxic solid, semi-solid or liquid filler, diluent, encapsulating material or formulation aid of any type. In the pharmaceutical compositions of the present description for oral, sublingual, subcutaneous, intramuscular, intravenous, transdermal, local or rectal administration, the active ingredient or the combination of active ingredients can be administered in a unit dosage form mixed with conventional pharmaceutical carriers to animals and human beings. Suitable unit dosage forms include oral administration forms such as tablets, gel capsules, powders, granules and oral suspensions or solutions, sublingual and buccal administration forms, aerosols, implants, subcutaneous, transdermal, topical, intraperitoneal, intramuscular, intravenous, subcutaneous, transdermal, intrathecal and intranasal administration forms and rectal administration forms. Typically, pharmaceutical compositions contain vehicles which are pharmaceutically acceptable for an injectable formulation. These can in particular be isotonic, sterile saline solutions (monosodium or disodium phosphate, sodium, potassium, calcium or magnesium chloride and the like or mixtures of these salts), or dry compositions, in particular freeze-dried, which by addition, depending on the case, of sterilized water or physiological serum, can be constituted as injectable solutions. Pharmaceutical forms suitable for injectable use include sterile aqueous solutions or dispersions; formulations comprising sesame oil, peanut oil or aqueous propylene glycol; and sterile powders for the extemporaneous preparation of sterile injectable solutions or dispersions. In all cases, the form must be sterile and must be fluid to the point that it can be easily tightened.

The pharmaceutical composition must be stable under the manufacturing and storage conditions and must be protected from the contaminating action of microorganisms such as bacteria and fungi. Solutions comprising the compounds of the disclosure in the form of a free base or pharmacologically acceptable salts can be prepared in water suitably mixed with a surfactant such as hydroxypropylcellulose. Dispersions can also be prepared in glycerol, liquid polyethylene glycols and mixtures thereof, and in oils. Under ordinary conditions of storage and use, these preparations contain a preservative to prevent the growth of microorganisms. The at least one bacterial strain of the species L. mucosae according to the present description can be formulated in a composition in a neutral form or in salt form. Pharmaceutically acceptable salts include acid addition salts (formed with the free amino groups of the protein) which are formed with inorganic acids such as, for example, hydrochloric or phosphoric acids, or organic acids such as acetic, oxalic, tartaric, mandelic, and similar acids. Salts formed with free carboxyl groups can also be derived from inorganic bases such as, for example, sodium, potassium, ammonium, calcium or ferric hydroxides, and from organic bases such as isopropylamine, trimethylamine, histidine, procaine and the like.

According to a particular embodiment, a bacterial strain according to the invention is included in an oral composition, and more particularly in an oral composition selected from the group consisting of a food product, a drink, a pharmaceutical product, a nutraceutical, a food additive, a food supplement, a dairy product and live biotherapeutic products (LBPs).

Within the meaning of the present description, the term “therapeutically effective amount” is an equivalent term which refers to the amount of a treatment (e.g., a prophylactic or therapeutic agent) which is sufficient to reduce the severity and/or duration of a disease, alleviate one or more of its symptoms, prevent the progression of a disease or cause the regression of a disease, or which is sufficient to result in the prevention of the development, recurrence, onset or progression of a disease or one or more of its symptoms or to enhance or improve the prophylactic and/or therapeutic effect(s) of another therapy (e.g., another therapeutic agent) useful for treating an illness. Typically, in a pharmaceutical composition according to the present description, the bacterial strains of the species L. mucosae are present in a quantity sufficient to induce a reduction in insulin resistance in the subject treated. Measurement of insulin resistance in a subject can be carried out using any technique known to the person skilled in the art. Preferably, measurement of insulin resistance in a subject is carried out by calculating the HOMA IR index, as illustrated in the examples. The HOMA IR method (for “HomeOstasis Model Assessment for Insulin Resistance”) was developed from mathematical modeling of the quantitative responses of the main organs of glucose metabolism. The HOMA IR index value is obtained using a plasma insulin or C-peptide value and fasting blood glucose (Sheen, 2007, Therapy, Vol. 62:311-318). The occurrence and/or level of insulin resistance can also be established by measuring fasting blood sugar, by measuring fasting insulin or by the OGTT test (for “Oral Glucose Tolerance Test” or “provoked hyperglycemia test”—cf, for example “Measurement of insulin resistance and glucose tolerance,” 2006, Haute Autorité de Santé (HAS), France).

In a composition according to the description, whether it is a nutritional or food composition, where appropriate a food supplement, or whether it is a pharmaceutical composition, the bacterium or bacteria can be present in various forms, for example in liquid form or in powder form. The bacterium or bacteria can be in freeze-dried form.

The quantity of bacteria of the species L. mucosae which is administered to the subject can be variable, depending on the physiological state of said subject, and in particular depending on the level of nutrient intake imbalance in relation to the nutrient requirements of said subject. The quantity of bacteria of the species L. mucosae to be administered to said subject can be easily adjusted by the person skilled in the art.

In preferred embodiments, whether the composition is a nutritional composition or a pharmaceutical composition, the composition comprises an amount of bacteria of the species L. mucosae which is suitable for daily intake, preferably daily oral intake, of at least 10³ colony forming units (or “CFU”).

In these preferred embodiments, the daily intake, in particular the daily oral intake, of bacteria of the species L. mucosae is at most 10¹³ colony forming units (or “CFU”),

In embodiments in which the composition additionally comprises other probiotic bacteria, the quantity of these probiotic bacteria is determined by the person skilled in the art based on his or her general knowledge. The quantity of these other probiotic bacteria can vary from 10³ to 10¹³ other probiotic bacteria.

In some embodiments, a composition according to the present description generally includes carriers or vehicles. “Carriers” or “vehicles” designate materials suitable for administration and include any material known in the prior art, such as, for example, any liquid, gel, solvent, liquid diluent, solubilizing agent or other, which is non-toxic and which does not interact with the components of the composition in a harmful manner. Examples of nutritionally acceptable carriers include, for example, water, saline solutions, alcohols, silicones, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, monoglycerides and diglycerides of fatty acids, fatty acid esters of petroleum, hydroxymethylcellulose, polyvinylpyrrolidone, etc.

In other embodiments of a composition according to the present description, said composition is in the form of a pharmaceutical composition comprising one or more pharmaceutically acceptable excipients.

Such a pharmaceutical composition can be presented in the form of packaging comprising a plurality of dosage units.

The term “dosage unit” is used in its conventional sense in pharmacy (e.g. a pill, a capsule, a tablet, the contents of an ampoule, etc.).

### Uses

GLP-1 is a peptide that has numerous metabolic effects in the host; in particular, it improves insulin secretion by the pancreas, promotes glucose tolerance, and therefore optimizes the energy and nitrogen metabolism of the host. The inventors have surprisingly shown that bacteria of the Limosilactobacillus mucosae type make it possible to increase the production of GLP-1 in vitro, and thus to prevent or limit the metabolic effects of diseases or conditions linked to a deficiency in the action of GLP-1: insulin resistance (for example type II diabetes), metabolic diseases, difficulty regulating food intake, regulation of transit disorders, etc.

As will appear in the description, a bacterial strain of the species L. mucosae according to the invention or a composition comprising it are mainly intended to prevent and/or treat:
(i) a disorder linked to dysregulation of insulin sensitivity and/or blood sugar, or
(ii) loss of muscle mass and/or muscle function.

According to a particular embodiment, the loss of muscle mass and/or muscle function is linked to insulin resistance.

These disorders are linked to a deficiency in the action of GLP-1.

A bacterial strain of the species L. mucosae according to the invention or a composition comprising it can in particular be useful in the prevention and/or treatment of disorders linked to transit or disorders of appetite regulation.

According to a particular embodiment, the bacterial strain is included in a composition comprising a physiologically acceptable medium, as defined above.

In the context of the present description, the term “prevent” or “prevention” refers to the reduction to a lesser degree of the risk or probability of occurrence of a given phenomenon, i.e. in the present invention, disorders requiring an increase in the level of GLP-1.

In the present text, the term “treat” or “treatment” refers to the relief or attenuation of pathological processes, or the relief or attenuation of one or more symptoms linked to a pathological process, and in particular to one of the disorders described in the application requiring an increase in the level of GLP-1.

The present description concerns the nutritional uses and therapeutic uses of a bacterial strain or of a composition as defined in the present application.

The compositional characteristics, including those of a nutritional composition and a therapeutic composition, including in various embodiments, are described in detail in the present description, which includes the amounts of active ingredient(s), especially the amounts of bacteria of the species L. mucosae included in these compositions.

(i) Disorders Linked to Dysregulation of Insulin Sensitivity and/or Blood Sugar

Dysregulation of insulin sensitivity, such as the occurrence of insulin resistance or insulino-resistance in particular, describes a situation in which liver, muscle and fat cells, for example, become resistant to insulin. There is then less glucose entering these cells, and it remains in the blood. In response to insulin resistance, pancreatic insulin-secreting cells tend to produce more insulin (hyperinsulinemia) and can eventually become exhausted. Insulin production then becomes insufficient and the blood glucose level becomes too high (hyperglycemia). Insulin resistance can also involve other types of metabolism regulated by insulin such as protein metabolism. In fact, insulin, combined with meals, is a major stimulator of muscle protein anabolism. Insulin resistance therefore contributes in this respect to muscle wasting due to a lack of anabolism in the muscle, particularly when eating a meal.

Blood sugar regulation is the process by which the level of glucose in the blood, known as blood sugar, is kept close to a value that is beneficial for the body. This regulation is part of the processes for maintaining homeostasis within the body. Normal fasting blood sugar in humans is statistically between 0.70 and 1.10 g/L. When blood sugar levels are abnormally higher or lower than this threshold, this is referred to as dysregulation.

The main disorders linked to dysregulation of insulin sensitivity and/or blood sugar according to the present text are prediabetes, type 1 diabetes and type 2 diabetes. They may or may not be caused by obesity.

However, the activation of receptors expressed at the level of insulin cells (B) and certain peripheral tissues by GLP-1 stimulates insulin secretion (insulinotropic effect) but also activates the transcription of the insulin gene, increases insulin biosynthesis and inhibits the release of glucagon (static glucagon effect).

“Prediabetes” refers to a physiopathological state characterized, in particular, by high blood sugar levels compared to normal, but below the threshold for defining type 2 diabetes. Fasting blood sugar levels are considered to be (i) normal between 0.70 and 1.10 g/l, (ii) indicative of prediabetes between 1.10 and 1.25 g/l and (iii) indicative of diabetes when >1.25 g/l. Prediabetes generally does not cause symptoms, but is often associated with obesity, dyslipidemia, and hypertension. It is a risk factor for cardiovascular disease. Prediabetes is characterized in particular by glucose intolerance. Stimulation of insulin and glucagon secretion by the pancreas by GLP1 controls glycemic excursions by reducing fasting blood sugar. GLP1 induces glucose uptake and hepatic storage and inhibits gluconeogenesis. Furthermore, in addition to increasing insulin secretion, GLP1 also plays a role in improving insulin sensitivity in many tissues, including muscle (Muller et al., 2019, Mol Metab).

“Type 1 diabetes” or “T1D” refers to a chronic disease which is characterized by the total absence of insulin production in an individual. The person living with type 1 diabetes therefore depends on daily insulin injections or an insulin pump to ensure survival. As explained above for prediabetes, GLP1 plays many roles in blood sugar control, including controlling the stimulation of insulin and glucagon secretion, glucose uptake and hepatic storage, inhibition of gluconeogenesis, and improvement of insulin sensitivity in many tissues.

“Type 2 diabetes” or “T2D” refers to a chronic disease which occurs when the pancreas does not produce enough insulin (blood sugar regulating hormone), or when the body is not able to use the insulin it produces effectively.

According to a particular embodiment, the disorder linked to the dysregulation of blood sugar is type 2 diabetes.

(ii) Loss of Muscle Mass and/or Muscle Function

According to a particular embodiment, the disorder to be prevented and/or treated is a loss of muscle mass and/or function, characterized in that the subject in need thereof is selected from among elderly subjects with sarcopenia, overweight and obese subjects with sarcopenic obesity on a diet, subjects with cachexia, in particular linked to cancer, inflammatory bowel disease or chronic obstructive pulmonary disease, immobilized subjects, diabetic or prediabetic subjects, “severe burn” subjects, subjects who are septic or have suffered a viral infection, convalescent and/or immobilized subjects, and subjects having undergone intestinal resection or presenting intestinal malabsorption.

Subjects who have lost muscle mass in the above-mentioned states are generally resistant to the anabolic action of the meal and/or the action of insulin. Although subjects experiencing significant muscle wasting are often insulin-resistant, not all insulin-resistant or prediabetic subjects are necessarily in a situation of muscle wasting, which is a phenomenon that can set in gradually over time, with muscle wasting and the development of insulin resistance being strongly linked metabolically (Daily and Park, 2022 Cells—DOI: 10.3390/cells11030338).

In fact, it is known that GLP-1 has advantageous properties making it possible to optimize the energy use of nutrients and limit muscle wasting (Hong et al., Journal of Cachexia, Sarcopenia and Muscle 2019; 10:903-918), particularly in insulin-resistant individuals (Massimo et al., The Impact of Glucose-Lowering Drugs on Sarcopenia in Type 2 Diabetes: Current Evidence and Underlying Mechanisms. Cells 2021, 10, 1958).

The loss of muscle mass can be measured by different technologies known to the person skilled in the art such as for example upper arm circumference, bioelectrical impedance, bioenergetic X-ray absorptiometry (DXA) and CT scanning (Mareschal et al., 2019, Journal of Clinical Medicine, doi: 10.3390/jcm8071040).

The loss of muscle function (strength, power, physical performance) can be measured by different technologies known to the person skilled in the art, such as the grip test (grip test using a dynamometer), 6 min walking speed, 30 s chair stand test, SPPB (Short Physical Performance Battery) test, which combines walking speed, balance, and sit-to-stand performance, and the “timed up and go” test (consists of getting up from the chair, walking 3 meters, turning around and sitting down again) (Beaudart et al 2019, Calcified Tissue International, doi: 0.1007/s00223-019-00545-w).

“Elderly subject,” within the meaning of the present description, refers to a human subject or a non-human mammalian subject, including a pet animal such as a dog or cat, which shows signs of senescence such as impaired metabolic functions (e.g. absorption, digestion, excretion, tissue and organ dysfunction), locomotor difficulties and reduced resistance to external aggressions. “Elderly subject” or “elderly person,” in the case of a human, refers to a subject aged 65 or over. “Elderly subject,” in the case of a canine, in particular a dog, refers to a subject (i) more than 12 years of age for a canine, in particular a dog, of small size or (ii) more than 9 years of age for a canine, in particular a dog, of medium size, or (iii) more than 7 years of age, for a canine, in particular a dog, of large size. “Elderly subject,” in the case of a feline, in particular a cat, refers to a subject over 13 years old.

Sarcopenia (a word from Greek which can be translated as “lack of flesh”), initially defined by a loss of skeletal muscle mass, is currently characterized by a loss of muscle mass associated with functional degradation (European Working Group on Sarcopenia in Older People (EWGSOP)—Cruz Jentoft et al, 2019, Age and Aging). After increasing up to the age of 20 to 30, muscle mass decreases by about 1% per year, even in healthy people. This process accelerates between the ages of 50 and 60. Sarcopenia impacts physical performance, promotes walking disorders and constitutes a factor in fragility and increased risk of dependence, particularly in the elderly. Note that sarcopenia affects not only the elderly but also insulin-resistant populations such as the obese, with such cases being referred to as sarcopenic obesity (Working Group of the European Society for Clinical Nutrition and Metabolism (ESPEN) and the European Association for the Study of Obesity (EASO)—Donini et al, 2022, Obesity Facts). This muscle wasting can also occur over shorter periods of time, particularly in intensely catabolic situations such as cancers and associated powerful treatments such as chemotherapy. In this case, one refers rather to cachexia (see below). Sarcopenia is associated with an increased risk of falls and fractures leading to immobilization which, in turn, aggravates sarcopenia. One thus observes a snowball effect amplifying the loss of muscle mass and function linked to age.

“Obesity” refers to a physiopathological state in which an individual presents, in particular, weight gain and excess adipose tissue, generally induced by an obesogenic diet, including, in particular, excessive consumption of high-calorie foods, genetic predispositions or insufficient or nonexistent athletic activity. An individual declared obese has a body mass index (BMI) greater than 30. The body mass index, according to an official definition from the World Health Organization (WHO), is an indicator of health risks associated with being overweight or underweight. BMI is calculated by dividing the individual's weight (in kilograms) by their height (in meters) squared. A BMI value is associated with a specific body shape according to the classification given by the WHO.

Unlike an “obese” individual, an “overweight individual” refers to an individual whose condition is not physiopathological. An overweight individual often also has excess fatty tissue. An individual is generally considered to be overweight when they have a body mass index (BMI) of between 25 and 30.

One speaks of sarcopenic obesity when an individual has both excess fat mass and a reduction in lean muscle mass. This clinical condition most often affects elderly people.

“Cachexia,” a profound weakening of the body, is characterized in particular by the wasting of adipose tissue and muscles. It is common in many diseases, including cancers that are difficult, if not impossible, to control or cure, and as specified above, occurs over much shorter periods of time (days to weeks). Cancers, particularly those of the pancreas and stomach, cause severe cachexia. Patients can lose 10 to 20% of their body mass. Cachexia can also be linked to other catabolic diseases, such as inflammatory bowel disease or chronic obstructive pulmonary disease, viral infection or sepsis.

It has been shown that decreased mobility is associated with a decline in muscle mass index and muscle strength. This is also true conversely, because the reduction in mobility and physical activity in the broad sense can worsen or cause sarcopenia. Immobilized subjects are therefore likely to develop a loss of muscle mass and/or function. In particular, in the context of the present text, it can involve subjects recovering and/or immobilized after a fall.

Subjects known as “severe burn” cases, as in the case of diseases inducing hypermetabolism (sepsis, viral attack, even cancer), are characterized by significant requirements for macro- and micronutrients to meet the increased needs linked to the pathology, in particular due to the healing process (in “severe burn victims”) and the inflammatory response (Knuth et al 2021, American Journal of Physiology. Cell Physiology). Furthermore, due to high inflammation, one generally sees resistance to the anabolic effect of meals and insulin resistance mentioned above as determinants in obesity and sarcopenia in elderly subjects.

In subjects with intestinal malabsorption, or who have undergone intestinal resection, GLP-1 can help optimize the use of nutrients. In fact, in patients who have undergone intestinal resection, one sees an increased production of GLP1 (Jeppesen et al 2000, Gut; Gillard et al 2017, Front Physiol) making it possible to adapt intestinal motility and optimize metabolism of the host with the lowest supply of nitrogen and energy nutrients. The repercussions will be particularly notable on the energy (fat mass) and nitrogen (lean mass and muscle mass) stocks of the subjects. An improvement in the intestinal secretion of GLP-1 in such subjects, specifically by administering a GLP-1 secreting probiotic, is therefore beneficial for them.

According to a particular embodiment, the subject to be treated, otherwise referred to as the subject in need thereof, is a subject presenting an indication for administration of GLP-1 receptor agonists. An individual with an indication for administration of GLP-1 receptor agonists is an individual suffering from a disorder or disease that can be treated or whose symptoms can be alleviated by administration to said individual of one or more GLP-1 receptor agonists.

GLP-1 receptor agonists are most often synthetic peptides whose polypeptide sequence is close to that of GLP-1 and which act by binding to GLP-1 receptors, generally with increased stability compared to endogenous GLP-1.

Examples of GLP-1 receptor agonists include exenatide, liraglutide, dulaglutide, semaglutide, tirzepatide, lixisenatide, albiglutide, exenatide QW, BI 456906 mazdutide (IBI362; LY3305677), retatrutide (LY3437943), danuglipron, exendin-4, or cotadutide.

According to a particular embodiment, the subject presenting an indication for administration of GLP-1 receptor agonists is selected from the list consisting of insulin-resistant subjects such as diabetic subjects, overweight subjects, in particular obese subjects, subjects who have undergone metabolic surgery, subjects seeking to regulate their appetite, subjects suffering from liver diseases, subjects suffering from cardiovascular diseases, in particular suffering from cardiovascular diseases linked to diabetes, subjects suffering from inflammation linked to diabetes and/or metabolic syndrome, subjects with sarcopenia, subjects with cachexia; and subjects suffering from neurodegenerative diseases.

“Metabolic surgery” refers to a type of surgery which aims to treat a metabolic disease by surgical methods. Metabolic surgery, also known as diabetes or sugar surgery, includes surgical procedures that control both diabetes and obesity. These can include surgical interventions to regulate appetite, gastric emptying, food preference and/or taste.

Liver diseases for which administration of GLP-1 agonists may be indicated are, for example, non-alcoholic steatohepatitis (NASH).

Cardiovascular diseases for which administration of GLP-1 agonists may be indicated include in particular atherosclerosis, myocardial infarction, stroke, or alteration of the lipid profile (total cholesterol, LDL cholesterol, HDL/LDL ratio).

Neurodegenerative diseases for which administration of GLP-1 agonists may be indicated include in particular Alzheimer's disease or Parkinson's disease.

According to a particular embodiment, the subject to be given preventive and/or therapeutic treatment is selected from elderly subjects with sarcopenia, overweight subjects, and obese subjects with sarcopenic obesity who are on a diet.

The description also describes a method of preventing and/or treating disorders requiring an increase in the level of GLP-1 in a subject in need thereof, said use being selected from (i) the prevention or treatment of a disorder linked to dysregulation of insulin resistance and/or blood sugar and (ii) prevention or treatment of loss of muscle mass and/or muscle function, characterized by comprising at least one step of administration of a bacterial strain of the species Limosilactobacillus mucosae.

Furthermore, the invention relates to the non-therapeutic use of a bacterial strain of the species L. mucosae according to the invention or a composition comprising it for maintaining or increasing muscle mass and/or function in a subject in need thereof, in particular in a subject selected from malnourished subjects, elderly subjects, in particular malnourished elderly subjects, and subjects engaged in intense physical exercise.

Within the meaning of the present description, “undernourished” refers to a “state of an organism in nutritional imbalance” (cf. “Diagnosis of undernutrition in children and adults-Method recommendations for clinical practice,” November 2019, National Health Authority and French Nutrition Federation), said imbalance being characterized by a negative energy and/or protein balance. This definition encompasses a plurality of situations that can lead to a state of undernutrition, such as a deficit in individual nutrient intake, an increase in expenditure or losses leading to an imbalance. This term also covers subjects with increased nutritional needs. The imbalance inherent in undernutrition leads to harmful effects on the body, accompanied by measurable changes in bodily functions and/or body composition, and these changes may be associated with a worsening of the progression of diseases which can affect the subject,

“Elderly subjects” are defined above.

“Intense physical exercise,” refers to physical activity requiring significant effort which causes breathlessness and acceleration of heart rate. Intense physical activity is characterized by a MET (Metabolic Equivalent of Task-1 MET corresponds to an oxygen intake of 3.5 mL per kilogram of body weight per minute) of greater than 6. The MET indicates the ratio between energy consumption during physical effort and energy consumption at rest. During physical exercise, particularly in insulin-resistant subjects, GLP-1 can constitute an interesting complementary strategy to help optimize blood sugar management and strengthen insulin sensitivity. Intense physical activity also leads to stimulation of GLP-1 production.

The present text also describes a method for maintaining or increasing muscle mass and/or function in subjects in need thereof, in particular in subjects selected from malnourished subjects, elderly subjects, in particular malnourished elderly subjects, and subjects engaged in intense physical exercise, comprising at least the step of administering to the subject in need thereof at least one bacterial strain of the species Limosilactobacillus mucosae.

Finally, the present invention relates to the strain of the species Limosilactobacillus mucosae deposited with the CNCM under accession number CNCM I-5661.

The invention is described below in more detail using the following examples, which are presented by way of illustration.

### EXAMPLE

### 1. Selection of Bacterial Strains Capable of Secreting GLP-1

Sixty-one Lactobacillus isolates originating from the feces of gnotobiotic animals previously transplanted with the feces of a patient with short bowel syndrome were tested. These lactobacilli have a strong tropism for the intestine, as these strains are abundant in the feces of patients with short bowel syndrome and are capable of colonizing the digestive tract of axenic rats.

The inventors carried out an initial morphological, phenotypic and genomic characterization by sequencing the 16S gene, dividing the isolates into 6 species, and selecting 1 to 2 isolates per species, for a total of 7 strains.

The 6 species are:
Lacticaseibacillus casei, (strain 1 and strain 2),
Lacticaseibacillus camelliae, (strain 3)
Ligilactobacillus salivarius, (strain 4)
Lacticaseibacillus rhamnosus, (strain 5)
Limosilactobacillus reuteri, (strain 6)
Limosilactobacillus mucosae, (strain CNCM I-5661, also called strain I-5661)

The strains were characterized by functional tests at INRAE in Aurillac (Unité Mixte de Recherche sur le Fromage Aurillac, UMR 0545).

All of the strains isolated above were incubated on intestinal neuroendocrine cells of SCT-1 mice. After incubation of 10⁹ bacteria with 2×10⁶ SCT-1 cells at 80% confluency for 4 hours at 37° C. and 5% CO₂, the cell supernatant was recovered and a peptide, GLP-1, was measured (by ELISA) in this supernatant.

The results are presented in FIG. 1.

Of all the strains tested, none demonstrated significant synthesis of GLP-1 in the culture medium, except for strain CNCM I-5661, which resulted in very significant production (P<0.05) of GLP-1 in the medium.

In fact, among all the strains from the same donor, only the strain of the species L. mucosae CNCM I-5661 specifically made it possible to increase the production of GLP-1 25 times. When lactic acid bacteria of the species Lacticaseibacillus casei, which were isolated from the same patient, were incubated under the same conditions, no release of GLP-1 was observed (results not presented here-see example 4 and associated results below).

This strain corresponds to the species Limosilactobacillus mucosae.

The control group corresponds to a culture of intestinal cells in a sterile medium (no production of GLP-1).

### Results of Induction of GLP-1 Secretion by STC-1 Cells

The STC-1 cells came from an ATCC collection (Reference STC-1, CRL-3254™) and were cultured and prepared for their co-incubation the day before the experiment. The bacteria, which had been frozen in a dry pellet, were thawed on the day of the experiment and taken up in a culture medium without FCS or antibiotics. They were prepared by successive dilution to meet the following MOI (Multiplicity of Infection): 1:10, 1:100 and 1:1000.

The STC-1 cells were washed and the medium containing the bacteria was then added. After 4 h of coculturing, the supernatants were collected and counted. The supernatants were then stored, as were the cells, at −80° C. for the subsequent determination of GLP-1. The quantity of GLP-1 was determined by ELISA according to the instructions of the supplier Assay Genie (ELISA GLP-1 Mouse (ref: MOFI00854)).

GLP-1 production is expressed in pg/ml.

The definitions of the terms used in Table 1 below are as follows:
Control: STC-1 cells in the presence of culture medium
MOI: 10 (number of bacteria added for one STC-1 cell)
C: indicates that the CNCM-15661 bacteria were cultured in MRS medium (supplier BD, reference: 288130, reconstituted according to the supplier's instructions), then centrifuged and frozen in the form of dry pellets. The dry pellets were then resuspended in the STC cell culture medium.
V: indicates that the CNCM-15661 bacteria were cultured in “veganMRS” medium (supplier BIOKAR, reference: BK176HA, reconstituted according to the supplier's instructions), then centrifuged and frozen in the form of dry pellets. The dry pellets were then resuspended in the STC-1 cell culture medium.
61C-10: incubation of STC-1 cells in the presence of the CNCM-15661 strain. MOI: 10 (10 times more bacteria than STC-1 cells)
61C-100: incubation of STC-1 cells in the presence of the CNCM-15661 strain. MOI: 100 (100 times more bacteria than STC-1 cells)
61C-1000: incubation of STC-1 cells in the presence of the CNCM-15661 strain. MOI: 1000 (1000 times more bacteria than STC-1 cells)

TABLE 1

| Experimental conditions | GLP1 (pg/ml) |
|---|---|
| Control | 0.0 |
| 61C-10 | 256.0 |
| 61C-100 | 454.2 |
| 61C-1000 | 1193.7 |
| 61V-10 | 253.6 |
| 61V-100 | 405.5 |
| 61V-1000 | 1240.5 |

The results shown in the table above indicate a dose effect of the strain and also show that the strain retains its functions even if it is cultivated in another medium.

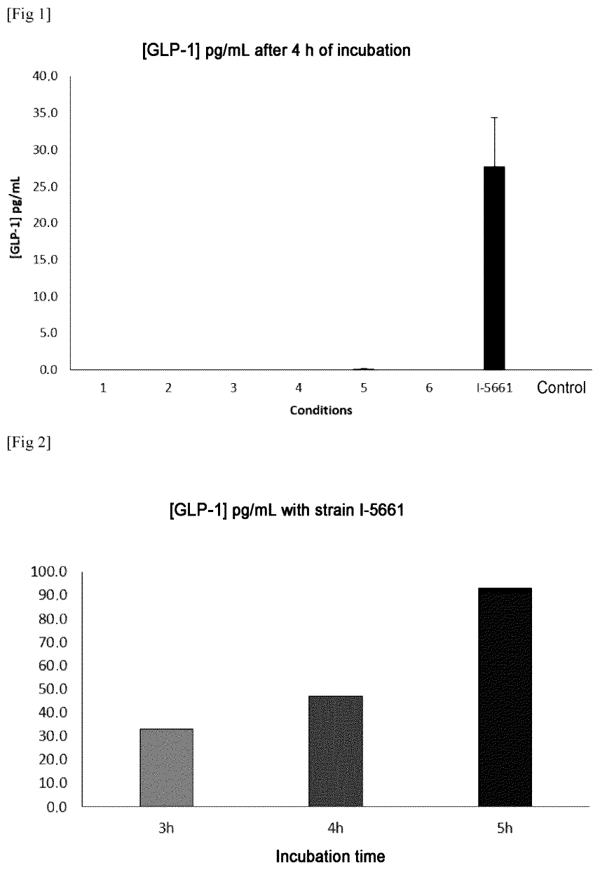

Conclusion: the presence of the CNCM-5661 strain induces the production of the enterohormone GLP-1 by STC-1 cells. This production is all the more important as the number of bacteria increases. This production is not observed in the presence of strains of the species Lacticaseibacillus casei which come from the same patient. The pro-GLP-1 effect of the CNCM I-5661 strain is not dependent on the culture medium used for the cultivation of the strain. Furthermore, the inventors showed that the production of GLP-1 increased as a function of the contact time between the bacteria of the CNCM I-5661 strain and the intestinal cells (see FIG. 2).

### 2-Capacity of the CNCM I-5661 Strain to Survive in the Intestinal Environment

To determine whether the CNCM I-5661 strain is capable of surviving in the intestinal environment, various tests were performed.
Tolerance of the bacteria for gastric (stomach) acidity

The bacteria were placed in an acidic medium (pH 2.5 and 3) for 45 and 90 minutes, corresponding to average residence times of the food bolus in the stomach before passing into the duodenum.

The bacteria showed very good resistance to the gastric medium (composition: HCl, pepsin, NaCl), with no significant difference between the incubation times and the two pH values tested (p>0.05, Fisher exact test).

The results are presented in Table 2 below.

TABLE 2

| | Gastric medium pH 2.5 | | Gastric medium pH 3 | |
|---|---|---|---|---|
| | 45 min | 90 min | 45 min | 90 min |
| Survival | 101.54 ± 0.97 | 102.92 ± 1.43 | 103.67 ± 1.29 | 104.27 ± 2.07 |
| percentage | | | | |

Tolerance of the bacteria for gastric acidity (bile salts)

The inventors also tested the survival of bacteria under conditions encountered downstream of the stomach which include the presence of bile salts. The survival of the CNCM I-5661 strain was therefore tested in the presence of bile salts after 1 h, 2 h, 3 h and 4 h of incubation at 37° C.

The bacteria showed very good resistance to bile salts, with no significant difference between the incubation times (p>0.05, Fisher exact test).

The results are presented in Table 3 below.

TABLE 3

| | 1 h | 2 h | 3 h | 4 h |
|---|---|---|---|---|
| Survival | 90.85 ± 2.28 | 88.74 ± 2.44 | 87.75 ± 1.46 | 82.67 ± 1.77 |
| percentage | | | | |

### 3—Capacity of the CNCM I-5661 Strain to Adhere to Intestinal Cells

Finally, in order to estimate the probiotic potential of the CNCM I-5661 strain, the inventors determined its capacity to adhere to human intestinal Caco-2 cells. Determination of the viable number of lactic acid bacteria adhering to intestinal cells was carried out after 3 hours of contact with Caco-2 cells at MOI (multiplicity of infection) values of 0.1, 1, 10 and 100.

The CNCM I-5661 strain showed a satisfactory adhesion percentage similar to that of other microorganisms, without any significant difference between the MOIs tested (p>0.05, Fisher exact test).

The results are presented in Table 4 below.

TABLE 4

| | Multiplicity of infection | | | |
|---|---|---|---|---|
| | 100 | 10 | 1 | 0.1 |
| Adhesion | 4.18 ± 0.65% | 3.90 ± 0.30% | 3.32 ± 0.07% | 1.84 ± 0.24% |
| percentage of | | | | |
| viable bacteria | | | | |

4—Extension of the Properties Described on the CNCM I-5661 Strain to the Entire Limosilactobacillus mucosae Species

The capacity of other strains of the species Limosilactobacillus mucosae to allow the secretion of GLP-1 by SCT-1 cells in culture was tested on other strains of the species Limosilactobacillus mucosae to determine whether the beneficial effect observed on the strain L. mucosae CNCM I-5661 is also applicable to other strains of the same species.

Three other strains were therefore tested: DSM 13345, DSM 13346, and DSM 102820. The strains CNCM I-5661 (as a positive control) and a strain of L. casei (negative control, which does not induce the synthesis of GLP-1) were also used:
Control: sterile cell medium;
L. casei strain (negative control) ˜5.10⁹ CFU/mL (strain A);
L. mucosae CNCM I-5661 (positive control) ˜5.10⁹ CFU/mL (strain B);
L. mucosae DSM 13345 (S32T) ˜2.10⁹ CFU/mL (strain C);
L. mucosae DSM 13346 ˜4.10⁹ CFU/mL (strain D);
L. mucosae DSM 102820 ˜5.10⁹ CFU/mL (strain E);
   Under the “diluted” conditions, strains B to E were diluted 1/10.

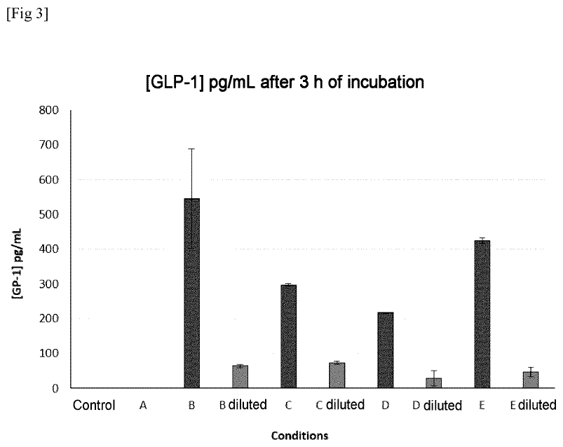

The results, presented in FIG. 3, show that in the presence of all strains of the species Limosilactobacillus mucosae tested, synthesis of GLP-1 by SCT-1 cells was observed. In addition, this production of GLP-1 was dose-dependent.

### 5—Effect of the CNCM I-5661 Strain on the Muscle Mass of a Fragile Aged Rat Model

To estimate the anabolic probiotic effect on GLP-1 targets, the inventors measured the capacity of the L. mucosae strain CNCM I-5661 according to the invention (shown to be capable of inducing the synthesis of GLP-1 by SCT-1 cells in culture) and a strain of the species L. casei outside the invention (not inducing GLP-1 production) on the muscle mass of a fragile aged rat model.

The inventors carried out an in vivo study over a period of 1 month on aged rats (20 months) according to the following groups:
group fed ad libitum (n=15);
group fed 75-80% of ad libitum intake (to mimic malnutrition in the elderly, one of the factors in fragility) (n=16);
group fed 75-80% of ad libitum intake supplemented each day with the bacteria CNCM I-5661 (10⁹ CFU) (n=13); and
group fed 75-80% of ad libitum intake supplemented each day with bacteria of the species Limosilactobacillus casei (10⁹ CFU) (n=16).

The animals were sacrificed at the end of the experiment, weighed, and the muscles of the hind leg (gastrocnemius, extensor digitorum longus, soleus, tibialis anterior) were excised and weighed. The sum of the weights of these 4 muscles was calculated and reduced to the weight of the animals.

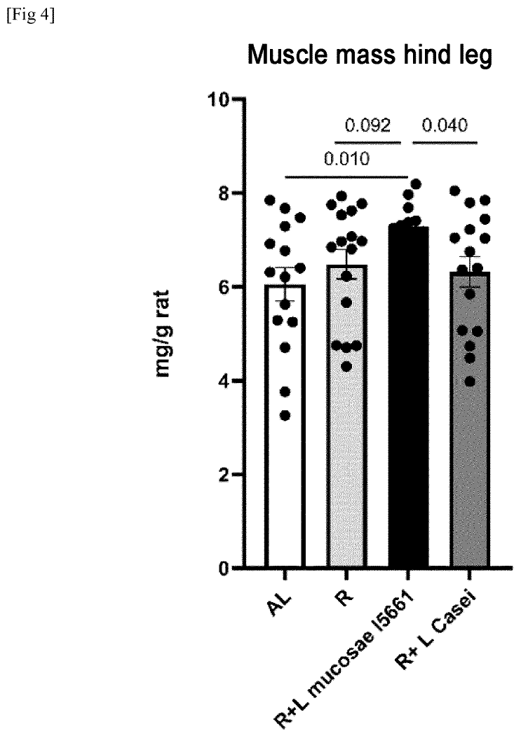

The results are presented in FIG. 4 and show that only supplementation with L. mucosae CNCM-15661 allowed restricted rats to increase their muscle mass relative to animals fed ad libitum (P=0.01) and restricted (trend, P=0.092).

Furthermore, muscle mass was similar in the AL, R and R+L casei groups, suggesting that unlike L. mucosae CNCM-15661, L casei did not increase muscle mass. This suggests a specific effect of Lactobacillus L. mucosae CNCMI-15661 of increasing muscle mass in the fragile aged rodent model, unlike other lactobacilli.

1. A method for preventing or treating a disorder requiring an increase in production of GLP-1 in a subject in need thereof, comprising providing the subject with a bacterial strain of the species Limosilactobacillus mucosae, or a lysate or a culture supernatant thereof, wherein said disorders being selected from (i) a disorder linked to dysregulation of insulin sensitivity and/or blood sugar and (ii) loss of muscle mass and/or muscle function.

2. The method as claimed in claim 1, wherein the subject in need thereof is a subject presenting an indication for administration of GLP-1 receptor agonists.

3. The method as claimed in claim 2, wherein the subject presenting the indication for administration of GLP-1 receptor agonists is selected from the list consisting of insulin-resistant subjects such as diabetic subjects, overweight subjects, in particular obese subjects, subjects who have undergone metabolic surgery, subjects seeking to regulate their appetite, subjects suffering from liver diseases, subjects suffering from cardiovascular diseases, in particular suffering from cardiovascular diseases linked to diabetes, subjects suffering from inflammation, in particular inflammation linked to diabetes and/or metabolic syndrome, subjects with sarcopenia, subjects with cachexia; and subjects suffering from neurodegenerative diseases.

4. The method of claim 1, wherein the disorder is a disorder linked to dysregulation of insulin sensitivity and/or blood sugar selected from prediabetes, type 1 diabetes and type 2 diabetes.

5. The method of claim 1, wherein the disorder is a loss of muscle mass and/or muscle function and in that the subject in need thereof is selected from elderly subjects with sarcopenia, overweight and obese subjects with sarcopenic obesity placed on a diet, diabetic or prediabetic subjects and/or subjects with cachexia, in particular linked to cancer, to inflammatory bowel disease or to chronic obstructive pulmonary disease, to immobilization, to “severe burn” status, sepsis or having suffered a viral infection, to convalescence and/or to immobilization, and/or to intestinal resection or intestinal malabsorption.

6. The method of claim 1, wherein the bacterial strain is selected from the strain of the species L. mucosae deposited with the CNCM under accession number CNCM I-5661, the strain of the species L. mucosae deposited with the DSM under accession number DSM 13345, the strain of the species L. mucosae deposited with the DSM under accession number DSM 13346, the strain of the species L. mucosae deposited with the DSM under accession number DSM 102820, or a mixture thereof, and in particular is the strain of the species L. mucosae deposited with the CNCM under accession number CNCM I-5661.

7. The method of claim 1, wherein the bacteria of the species L. mucosae are in a living or dead form.

8. The method of claim 1, wherein the bacterial strain is included in a composition comprising a physiologically acceptable medium, in particular in an oral composition, and more particularly in an oral composition selected from the group consisting of a food product, a beverage, a pharmaceutical product, a nutraceutical, a food additive, a food supplement, a dairy product and live biotherapeutic products (LBPs).

9. The method of claim 8, wherein the composition further comprises one or more other probiotic bacterial strains, in particular selected from the species Bifidobacterium longum, Bifidobacterium lactis, Bifidobacterium breve, Bifidobacterium infantis, Bifidobacterium adolescentis, Lactobacillus acidophilus, Lactobacillus casei, Lactobacillus salivarius, Lactobacillus johnsonii, Lactobacillus salivarius, Lactococcus lactis, Enterococcus faecium, Enterococcus faecalis, Saccharomyces cerevisiae, Saccharomyces boulardii, Faecalibacterium prausnitzii, Akkermansia muciniphila, Blautia faecis, Faecalibacterium prausnitzii, Streptococcus Thermophilus or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus johnsonii NCC533 (CNCM 1-1225), Enterococcus faecium SF 68 (NCC2768; NCIMB10415), Lactobacillus casei (CNCM I-5662 and CNCM I-5663), Streptococcus thermophilus (CNCM I-5334) and combinations thereof.

10. The method of claim 8, wherein the composition is free of any other bacteria of the Lactobacillus species.

11. The method of claim 8, wherein the composition further comprises one or more prebiotics.

12. A non-therapeutic method for maintaining or increasing muscle mass and/or function in a subject in need thereof, comprising providing the subject with the bacterial strain of the species Limosilactobacillus mucosae, or a lysate or a culture supernatant thereof as defined in claim 6.

13. A bacterial strain Limosilactobacillus mucosae deposited with the CNCM under accession number CNCM I-5661.

14. The method for claim 1 wherein the bacteria of the species L. mucosae are in a living form.

15. The method of claim 12 wherein the subject is selected from the group consisting of malnourished subjects, elderly subjects, and subjects engaged in intense physical exercise.
